(19) United States
(12) Patent Application Publication
(10) Pub. No.: US 2022/0061572 A1
(43) Pub. Date: Mar. 3, 2022

(54) SANITARY DELIVERY CONTAINER SYSTEM

(71) Applicant: Porchboxx Inc., Tampa, FL (US)

(72) Inventor: CLYDE RICHARD SNODGRASS, Tampa, FL (US)

(21) Appl. No.: 17/003,485

(22) Filed: Aug. 26, 2020

(51) Int. Cl. A47G 29/14; G06Q 30/00; G06Q 50/28; G06Q 10/10; G07C 9/00; G06K 7/14; E05G 1/10; E05G 1/12; E05B 65/00; A61L 2/26; A61L 2/10; A61L 2/20

(52) U.S. Cl. CPC A47G 29/141; F25D 31/005; G06Q 50/28; G06Q 10/107; G07C 9/00182; G06K 7/1413; G06K 7/1417; E05G 1/10; E05G 1/12; E05B 65/0075; A61L 2/26; A61L 2/10; A61L 2/202; A47G 2029/147; A47G 2029/145; A47G 2029/149; G06Q 30/0185

(57) ABSTRACT

A device and method for securely delivering a package are provided. The device may be a container for receiving packages, and may include a body configured to receive a package, a door coupled to the body and having a locking mechanism configured to prevent the door from opening when engaged, a controller configured to receive a signal corresponding to the package, and to unlock the locking mechanism in response to the signal, and a sanitization device configured to sanitize at least one of the package and contents of the package by emitting at least one of a gas and a light configured to kill one or more of bacteria, viruses, and other pathogens. The method for securely delivering a package may include storing information corresponding to the package in a memory of the container, receiving a signal indicating that the package is ready for delivery to the container, unlocking a locking mechanism of the container in response to receiving the signal, and activating a sanitization device to sanitize the package after the package has been placed inside the container.

### FIELD

The present disclosure relates generally to a container for receiving delivered packages, and more specifically to a container that provides enhanced security to prevent theft as well as sanitize packages.

### BACKGROUND

Over the last several years, more and more consumers have been purchasing products online, as opposed to patronizing brick and mortar stores. Some of the purchases are made directly from a retailer's web site, whereas others are made from online marketplaces such as Amazon® that allow many sellers to sell their products online from the same website. Unlike purchases from brick and mortar stores, online purchasing of course requires delivering the purchased products to the consumer. Some retailers have developed their own delivery services that include a network of warehouses, delivery trucks and drivers, and software used to track the status of deliveries. Other retailers use third-party vendors, such as UPS® or FedEx® to delivery purchased products to the consumer's home. In both cases, products are often left on the consumer's front porch or near the front door of the consumer's home, which leaves the packages susceptible to theft and/or damage from weather.

In addition, with recent spread of the COVID-19 pandemic, and social distancing measures intended to prevent the virus' spread, many delivery drivers that might otherwise knock on the door to hand-deliver the purchased product to the consumer are merely leaving the product near the consumer's front door to avoid interpersonal contact that may spread the virus. Thus, the practice of leaving packages outside the consumer's front door has only increased with the onset of the pandemic. Moreover, many consumers are concerned that the packages themselves may contain the virus. With delivery drivers handling many packages and potentially interacting with many consumers, there is a concern that delivery drivers may unknowingly leave traces of the virus on the outside of the packages as they are handling the packages for delivery. Accordingly, there is a need for a secure container for receiving packages in a secure and sanitary manner.

### SUMMARY

According to an aspect of one or more exemplary embodiments there is provided a container for receiving packages that includes a body configured to receive a package, a door coupled to the body, the door having a locking mechanism configured to prevent the door from opening when engaged, a controller configured to receive a signal corresponding to the package, and to unlock the locking mechanism in response to the signal, and a sanitization device configured to sanitize at least one of the package and contents of the package by emitting at least one of a gas and a light configured to kill one or more of bacteria, viruses, and other pathogens. The sanitization device may be an ultraviolet light configured to emit light having a wavelength configured to kill one or more of bacteria, viruses, and other pathogens, and/or an ozone generator configured to generate ozone gas configured to kill one or more of bacteria, viruses, and other pathogens.

The container may also include a motion sensor configured to detect motion within the body of the container. The controller may be configured to deactivate the sanitization device in response to the motion sensor detecting motion within the body of the container. The container may also include an alarm, wherein upon receipt of the signal corresponding to the package, the controller causes the alarm to emit a sound or vibration to alert a deliver person to the presence of the container.

The container may also include a memory chip configured to store an identification number corresponding to package to be delivered to the container, and a touchscreen configured to receive an input number. The signal to unlock the locking mechanism may be generated based on the input number matching the identification number stored in the memory chip. The container may also include a code reader configured to decode information encoded in a code on a package, such that the signal to unlock the locking mechanism is generated based on the information decoded by the code reader matching identifying information stored in the memory chip.

The controller may receive the signal to unlock the locking mechanism by receiving a webhook from a server. The server may generate the webhook in response to a remote scanning device decoding information encoded in a code on a package, and the decoded information matching identifying information stored in a delivery tracking system for a package to be delivered to the container. Alternatively, the server may generate the webhook in response to receiving an email sent to a unique email address corresponding to the container indicating that a package is ready for delivery to the container. The email may be generated by a delivery tracking system in response to receiving a signal from a remote scanning device containing information decoded from a code located on the package that matches identifying information stored in the delivery tracking system for a package to be delivered to the container.

The container may also include at least one of a heating element configured to increase an interior temperature of the container and a cooling element configured to decrease the interior temperature of the container. The signal to unlock the locking mechanism may include a temperature signal to activate the heating element or cooling element. The controller may be configured to activate at least one of the heating element and cooling element based on the temperature signal.

According to an aspect of one or more exemplary embodiments there is provided a method of securely delivering a package to a container including the steps of storing information corresponding to the package in a memory of the container, receiving a signal indicating that the package is ready for delivery to the container, unlocking a locking mechanism of the container in response to receiving the signal, and activating a sanitization device to sanitize the package after the package has been placed inside the container. The method may also include determining whether motion occurs within the container, such that the activating of the sanitization device depends on whether motion is detected within the container. Activating the sanitization device may include emitting at least one of an ultraviolet light and an ozone gas configured to kill one or more of bacteria, viruses, and other pathogens.

The method may also include activating an alarm to emit a sound or vibration in response to receiving the signal indicating that the package is ready for delivery to the container. Receiving a signal indicating the package is ready for delivery may include receiving identifying information related to the package via an input device of the container. The method may also include comparing the received identifying information to the information corresponding to the package stored in the memory of the container, and unlocking the locking mechanism based on whether the received identifying information corresponds to the information stored in the memory of the container.

The input device may include a code reader, and the identifying information related to the package may include encoded information on the package that is decoded by the code reader. The step of receiving a signal indicating the package is ready for delivery may include receiving a webhook from a server, which may generate the webhook in response to a remote scanning device decoding information encoded in a code on the package, and the decoded information matching identifying information stored in a delivery tracking system for a package to be delivered. Alternatively, the server may generate the webhook in response to receiving an email sent to a unique email address corresponding to the container indicating that a package is ready for delivery to the container. The email may be generated by a delivery tracking system in response to receiving a signal from a remote scanning device containing information decoded from a code located on the package that matches identifying information stored in the delivery tracking system for a package to be delivered to the container. The method may also include activating at least one of a heating element and a cooling element based on receiving the signal indicating that the package is ready for delivery to the container.

### BRIEF DESCRIPTION OF DRAWINGS

FIG. 1 shows an external view of a container according to an exemplary embodiment.

FIG. 2 shows the interior of the container according to the exemplary embodiment of FIG. 1.

FIG. 3 shows a block diagram of a communication system that allows communication between the container and multiple package tracking systems according to an exemplary embodiment.

FIG. 4 shows a flow chart illustrating a process by which the container is registered and configured to receive packages according to an exemplary embodiment.

FIG. 5 shows a flow chart illustrating a process by which the container is notified that a package is to be delivered, according to an exemplary embodiment.

FIG. 6 shows a flow chart illustrating a process for delivery temperature-sensitive packages according to an exemplary embodiment.

### DETAILED DESCRIPTION OF EXEMPLARY EMBODIMENTS

Reference will now be made in detail to the following exemplary embodiments, which are illustrated in the accompanying drawings, wherein like reference numerals refer to like elements throughout. The exemplary embodiments may be embodied in various forms without being limited to the exemplary embodiments set forth herein. Descriptions of well-known parts are omitted for clarity.

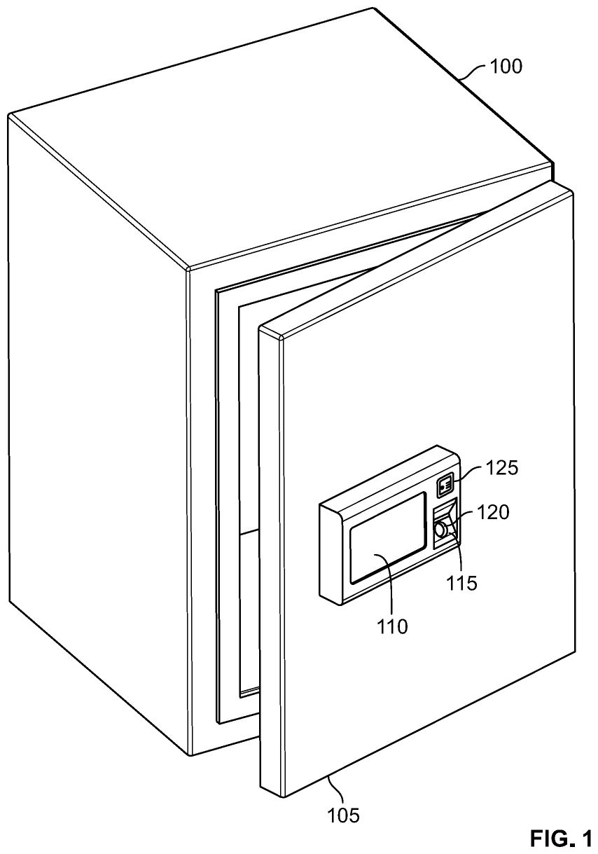

FIG. 1 shows an external view of a container according to an exemplary embodiment. Referring to FIG. 1, the container of the exemplary embodiment may include a body 100 and a door 105 that is movably coupled to the body 100. In the exemplary embodiment of FIG. 1, the body 100 and the door 105 are shown as having a rectangular shape, however this shape is merely exemplary as the body and the door may take any shape that allows the container to hold a package and allows the door to open and close. The container of the exemplary embodiment may also include a display screen 110 that is configured to display information regarding the container and receive signals for controlling the container. In the exemplary embodiment shown in FIG. 1, the display screen 110 is a touch screen that is configured to receive input instructions from a user by using a stylus or one or more of the user's fingers. Alternatively, the display screen 110 may include one or more buttons for selecting between options displayed on the screen. The exemplary container of FIG. 1 may also include a camera 115 configured to capture photographs and/or video of the area around the container. The camera 115 may be WiFi-enabled so that the camera 115 can transmit video or image data wirelessly using, for example, a local area network (LAN), wide area network (WAN), etc. The camera 115 may also be coupled to a controller (discussed below) that may be configured to communicate via WiFi to transmit image and/or video data captured by the camera 115. The exemplary container of FIG. 1 may be configured to be coupled to an AC power supply to provide power to the screen 110, the camera 115, the controller (not shown), and other components discussed below. Alternatively, the container may be configured to receive power from other power sources, such as batteries, solar panels, etc. The exemplary container may also include a motion detector 120 that is configured to detect motion near the container. For example, the motion detector 120 may be configured to detect motion within twenty (20) feet of the container, however a different distance may be used. The motion detector 120 may be coupled to the camera 115, which may be configured to begin capturing video data and/or still images upon receiving a signal from the motion detector 120 that motion has been detected near the container. According to an alternative exemplary embodiment, rather than relying on an external motion detector 120, the camera 115 may include an internal motion detector that causes the camera 115 to begin capturing video data and/or still images upon detecting motion near the container. The exemplary container of FIG. 1 may also include a code reader 125 configured to decode various types of codes placed on packages that are to be delivered. For example, the code reader 125 may be an optical scanner that emits light to detect and decode a bar code or QR code. The code reader 125 may also be a camera that captures an image of a barcode or QR code, and includes image processing circuitry to decode information contained within the barcode or QR code. Although the exemplary embodiment references barcodes and QR codes, other codes and decoding apparatuses may be used.

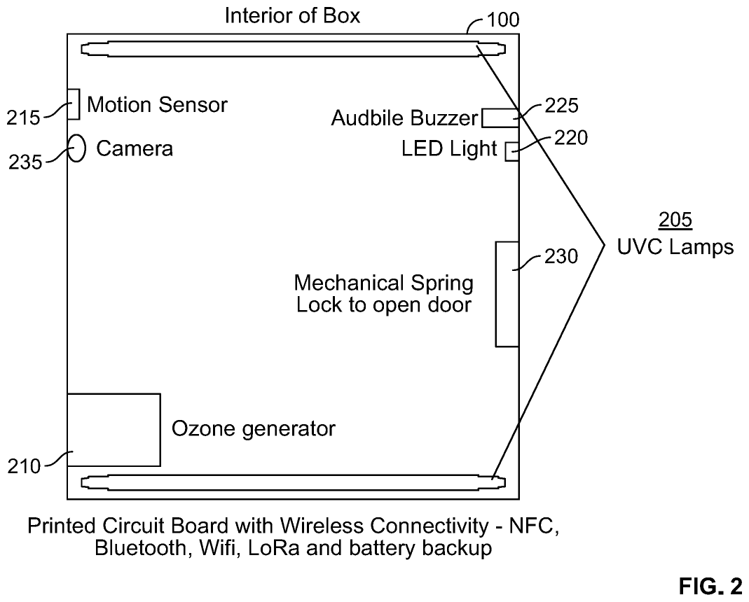

FIG. 2 shows the interior of the container according to the exemplary embodiment of FIG. 1. Referring to FIG. 2, the container may include one or more light emitting devices 205, such as ultra violet lamps, that are configured to emit light that kills viruses, bacteria, and/or other pathogens. For example, the light emitting devices may emit shortwave ultraviolet or ultraviolet-C (UV-C) light having a wavelength of approximately 200 nm to 300 nm, which has been shown to kill bacteria, viruses, and other pathogens.

The container according to the exemplary embodiment may also include an ozone generator 210, which generates ozone (O₃) at a concentration configured to kill viruses, bacteria, and/or other pathogens. For example, ozone generator 210 may produce an ozone concentration of 4 PPM (parts per million) to kill a variety of pathogens, however other concentrations may be used. Although the exemplary embodiment is described using ozone gas, different types of gas may be used to kill pathogens within the container. The container may also include a motion sensor 215 that is configured to detect motion within the container. The motion sensor 215, along with the ozone generator 210, may be coupled to a controller, which may be mounted on a printed circuit board (PCB) (not shown). The motion sensor 215 may be configured to output a signal to the controller indicating that motion has been detected within the container. The controller may be configured to transmit, upon receipt of a signal from the motion sensor 215 indicating motion within the container, a signal to the ozone generator 210 to stop generating ozone gas to avoid harming a living object that may have inadvertently entered the container. Alternatively, the controller may be configured to activate the ozone generator 210 upon receipt of a signal from motion sensor 215 that no motion has been detected. The container may also include an internal camera 235 configured to capture image or video data of the interior of the container. For example, the internal camera 235 may be coupled to the motion sensor 215 and configured to begin capturing image and/or video data when the motion sensor 215 detects motion within the container.

The container may also include a mechanical spring lock 230 that is configured to lock and unlock the door 105. The mechanical spring lock 230 may be electrically coupled to the controller and configured to lock and unlock the door 105 based on lock and unlock signals received from the controller. The container may also include an audible buzzer 225 and LED light 220. The audible buzzer 225 may be electrically coupled to the controller to emit a sound when a package is to be delivered to alert a delivery person to the presence of the container. The LED light 220 may also be electrically coupled to the controller, and be configured to output a light to notify the delivery person of the container's presence when a package is to be delivered. Although the exemplary embodiment includes an audible buzzer 225 and LED light 220, any device or devices that emit sound or light, cause vibration, or otherwise draw attention to the container upon delivery of a package may be used. The mechanical spring lock 230 may be coupled to the controller in such a way that the controller can determine whether the mechanical spring lock 230 is locked or unlocked. For example, the mechanical spring lock 230 may include a sensor that detects whether the mechanical spring lock 230 is unlocked or locked. Alternatively, the mechanical spring lock 230 may be coupled to the controller via a circuit that transmits a signal to the controller depending on whether the mechanical spring lock 230 is locked or unlocked.

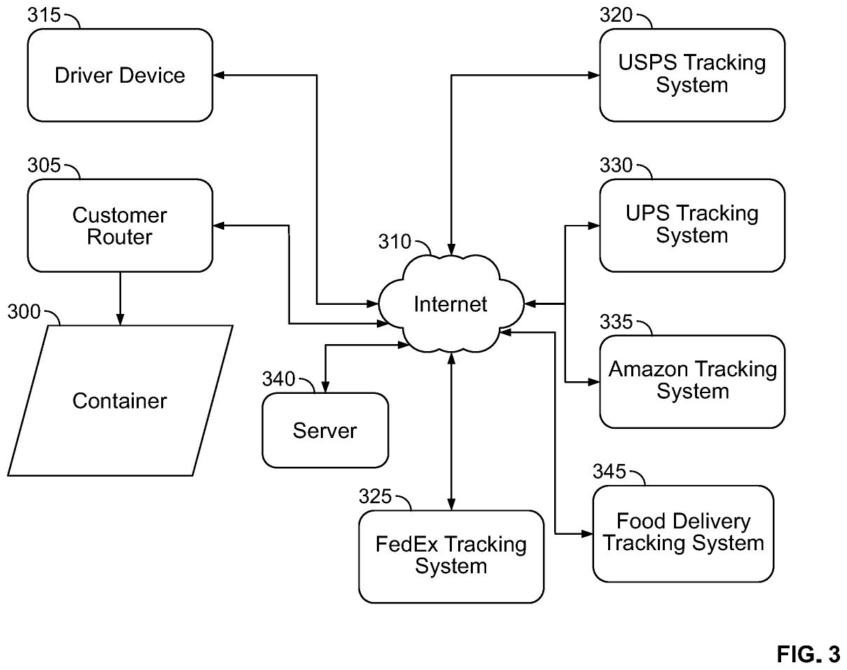

FIG. 3 shows a block diagram of a sanitary delivery container system according to an exemplary embodiment. Referring to FIG. 3, the system may include a container 300, such as the exemplary container shown in FIG. 1, which may be located at a customer's residence or business. The container 300 may be communicably coupled to the customer's router 305, which may be located at the customer's residence or business with the container 300. The router 305 may be communicably coupled to the Internet 310, which may be communicably coupled to a driver device 315 and various courier service tracking systems such as a United States Postal Service (USPS) tracking system 320, a FedEx® tracking system 325, a United Parcel Service (UPS®) tracking system 330, and an Amazon® tracking system 335. The exemplary system of FIG. 3 may also include a server 340 that is configured to communicate with the container 300 via the Internet 310 and router 305, as will be explained in more detail below.

The various courier service tracking systems 320, 325, 330 and 335 may be configured to record the status and location of a package as it is transported from the sender's location to the recipient's location. For example, when the package is initially picked up from the sender's location or dropped off by the sender at the courier service's receiving location, a label having a unique identifier, e.g., tracking number, barcode, QR code, etc., may be affixed to the package. An entry may be created in the courier service tracking system that associates the package with the unique identifier, and includes the time and location when the courier service received the package for delivery.

As the package is transported to the ultimate recipient location, the package may pass through multiple facilities of the courier service. At each stop along this journey, the time and location of the package may be updated in the courier service tracking system by using the unique identifier. For example, a package may be received at an initial location and subsequently transported to a nearby airport where the package will be loaded onto an airplane to be transported to a courier service facility closer to the ultimate delivery location. Upon arriving at the airport, the unique identifier may be scanned by a scanning device or manually entered into a terminal at the airport location, which transmits the updated time and location information associated with the unique identifier via Internet 310 to the appropriate courier service tracking system 320, 325, 330, or 335. This process repeats at each stop in the delivery process so that the various courier service tracking systems 320, 325, 330, or 335 are updated with new time and location information each time the package reaches a new location. Eventually, the package may be loaded onto a truck or other vehicle for delivery to the final delivery location. The driver may have a driver device 315 that is configured to scan the unique identifier and/or receive manual entry of the unique identifier by the driver. Upon scanning or receiving the unique identifier via manual entry, the driver device 315 may transmit, via Internet 310, updated status information to the corresponding courier service tracking system 320, 325, 330, or 335 indicating that the package has been delivered at the delivery location. The corresponding courier service tracking system 320, 325, 330, or 335 may be configured to transmit a notification via email, text message, etc. to the recipient that the package has been delivered. The recipient's email address and/or mobile phone number may be entered into the courier service tracking system when the package is first picked up for delivery, or may be provided to the courier service separately to be associated with deliveries to the recipient's delivery address. As explained further below, the courier service tracking system 320, 325, 330 or 335 that is tracking the package to be delivered may transmit a delivery notification, via Internet 310, to the container 300 via router 305.

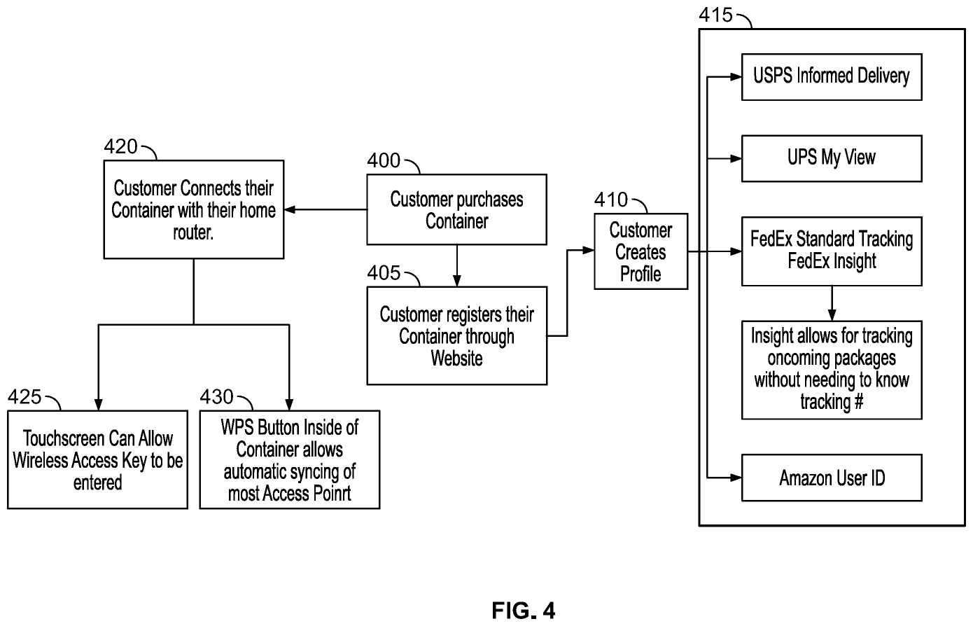

FIG. 4 shows a flow chart illustrating a process of configuring a delivery container according to an exemplary embodiment. At step 400, the customer purchases the container, and at step 405 the customer accesses a registration website via the World Wide Web to register the container. At step 410, the user creates a profile that contains bibliographic information about the customer, such as the customer's name and the address where the container will be located for receiving packages. The PCB may include a wireless chip (not shown) configured to communicate wirelessly with other networked devices via Wi-Fi or other wireless communication protocols. The wireless chip contains a unique media access control (MAC) ID that is used to allow the wireless chip (and thereby the container) to communicate with other networked devices via Wi-Fi or other known wireless communication protocols via the Internet. When creating the profile for the container in step 410, the user may enter the MAC ID associated with the wireless chip included in the container so that the MAC ID is associated with the physical location of the container, which is also contained in the profile. The profile may be stored in server 340 that is configured to communicate with the container using the container's MAC ID. Alternatively, the user may include an alternative identification number, such as a product serial number, rather than the MAC ID, in the profile. The server 340 may include a database that correlates product serial numbers with the MAC ID of the wireless chip included in the container bearing the serial number. The server 340 can therefore communicate with the container 300 by obtaining the MAC ID that corresponds to the serial number provided by the customer. According to an exemplary embodiment, step 410 may include the step of automatically generating a unique email address for the customer. The unique email address may be associated with the other bibliographic information contained in the profile, such as the customer's name and the address where the container is located.

At step 415, the server 340 may provide the MAC ID of the container to the courier service tracking systems 320, 325, 330, and 335. Each of the tracking systems may include informed delivery profiles for a customer that includes the customer's address and contact information needed to notify the customer of deliveries. When creating the customer profile for the customer's container, the customer may identify certain courier services that the customer uses, and may include login credentials needed to update the informed delivery profiles for each courier service. The server 340 may transmit a request to the various courier service tracking systems 320-335 that includes the login credentials provided to the server 340 when creating the container profile. The request may also include the MAC ID of the container 300, which may be added to the informed delivery profiles of the various courier services, once the courier service tracking system has verified the login credentials provided and identified the informed delivery profile associated with the login credentials. Alternatively, the customer may manually update his or her informed delivery profile with each of the courier services to include the MAC ID of the container. According to an exemplary embodiment in which a unique email address is created as part of the customer's profile, the unique address may be transmitted in the request to the courier service tracking systems and added to the respective informed delivery profiles.

At step 420, the customer supplies power to the container 300, for example, by plugging the container 300 into a standard electrical outlet, inserting batteries into the container, or using any other power source, so that the user can configure the container 300 to communicate with the customer router 305, which may be accomplished via a Wi-Fi connection, for example. In step 425, the customer may use the touchscreen 110 to input a wireless access key or password that is needed to communicate with the router 305. Alternatively, in step 430, the customer may press a Wi-Fi Protected Setup (WPS) button provided on the container in order to establish a connection with the router 305 depending on the security protocol (e.g., WPA, WPA2) used by the router 305.

Once container 300 has been connected to the router 305, and the MAC ID of the container 300 is added to the informed delivery profiles for the various courier services, the courier services tracking systems 320, 325, 330, 335 can communicate via Internet 310 and router 305 with the container 300. For example, if a customer purchases a product from an online retailer, and the online retailer uses FedEx® to ship the product to the customer, the FedEx tracking system 325 may create a new tracking entry that is associated with the customer's informed delivery profile, and which includes a unique tracking number and/or other unique identifier (e.g., bar code, QR code, etc.). The FedEx tracking system 325 may transmit a notification of the new tracking entry to the container 300 using the container's MAC ID that was previously associated with the customer's informed delivery profile. The notification may include the unique tracking number/identifier for the package, as well as other information relating to the package (e.g., identity of the sender, originating location, etc.). The container 300 may receive similar notifications from other courier service tracking systems 320, 330, 335 when a package is to be delivered to the container 300. The container 300 may include a memory chip configured to store information related to each delivery intended for the customer, including tracking numbers/identifiers for each package. According to an exemplary embodiment in which a unique email address is created for each customer profile and added to the respective informed delivery profiles for the various courier services tracking systems, when a new delivery is created in one of the courier service tracking systems 320, 325, 330, or 335 and is associated with the customer's informed delivery profile, the courier service tracking system may generate and transmit an email to the unique email address. The email may include a unique tracking number/identifier for the package, as well as other information relating to the package.

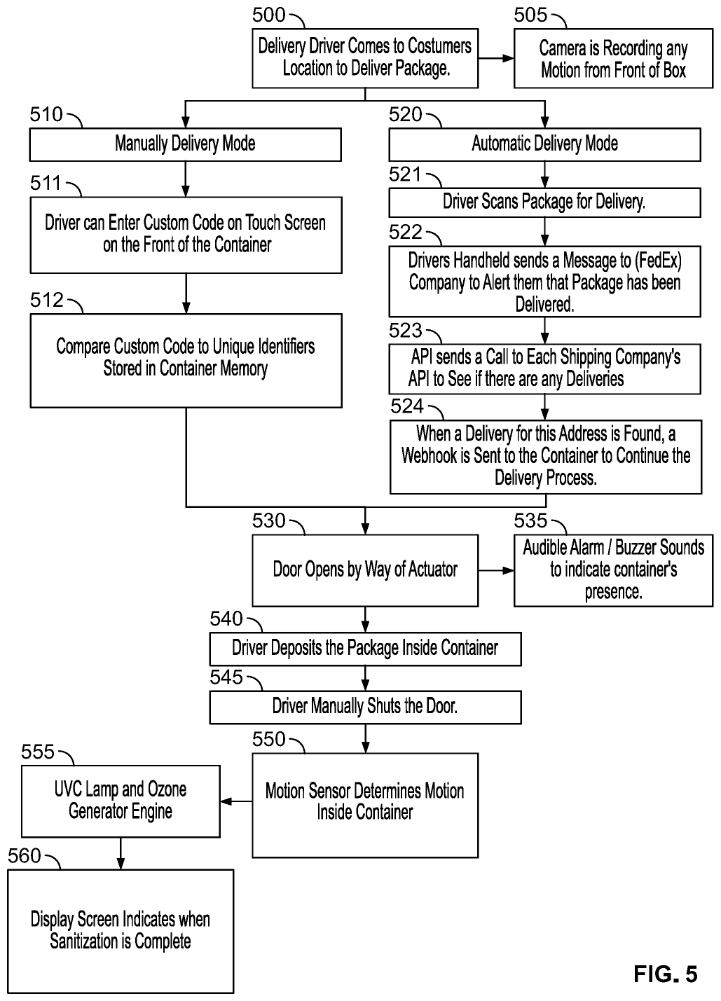

FIG. 5 shows a flow chart illustrating the process by which the container 300 is notified that a package is to be delivered, according to an exemplary embodiment. At step 500, the delivery driver arrives at the container's location to deliver a package. At step 505, the container's external motion detector detects the driver's motion as the driver approaches the container, and activates the container's camera in response to detecting the driver's motion. The delivery driver may then determine whether to deliver the package according to the manual delivery mode 510 or the automatic delivery mode 520.

If the delivery driver chooses the manual delivery mode 510, at step 511 the driver may enter a custom code using screen 110, which may be a touch screen. For example, the custom code may be a tracking number assigned to the package, or a portion of the tracking number. At step 512, the container may compare the custom code entered by the driver with a list of unique identifiers stored in the memory of the container, which were received from the courier service tracking systems 320, 325, 330, 335. If the custom code entered by the driver does not match one of the unique identifiers stored in the memory of the container, the screen 100 of the container may indicate that the code is incorrect, and ask the driver to re-input the code. If the custom code entered by the driver matches one of the unique identifiers stored in the memory of the container, in step 530 the controller causes the mechanical spring lock 230 to unlock so that the door 105 can be opened.

If the driver chooses the automatic delivery mode 520, in step 521, the delivery driver may scan a QR code, bar code, or other type of identifying symbol that is associated with the package that is to be delivered. The scanning step may be done using various types of scanners, including handheld scanners or other mobile devices typically carried by delivery drivers, as one of ordinary skill in the art would understand. At step 522, the driver's device 315 transmits a notification to the corresponding courier delivery service tracking system 320, 325, 330, 335 indicating that the package has been scanned as ready for delivery. At step 523, an application programming interface (API) of the container 300 transmits a call to each courier service tracking system 320, 325, 330, 335 to determine if there are any deliveries for the address where the container is located. The API call may include information such as the location of the container, the name of recipient, etc. Upon receiving the call from the API of the container, the courier service tracking systems 320, 325, 330, 335 query their respective databases to determine whether the database includes an entry for a delivery to the address contained within the request from container's API. If one of the courier service tracking systems 320, 325, 330, 335 determines that a delivery for the specified address is found in its database, it transmits a webhook in step 524 to the container API indicating that a package for delivery to the specified address has been identified. Once the webhook indicating a package has been identified for delivery to the specified address has been received, in step 530 the controller causes the mechanical spring lock 230 to unlock so that the door 105 can be opened.

Alternatively, according to another exemplary embodiment, after the driver's device 315 transmits a notification to the corresponding courier delivery service tracking system 320, 325, 330, 335 indicating that a package is ready for delivery in step 522, the delivery service tracking system may generate and transmit an email to a unique email address that was generated when creating the customer's profile, as described above. The email may indicate that a delivery is ready for the container 300 associated with the unique email address, and may contain information such as a unique tracking number/identifier, destination address, and other information relating to the package to be delivered. The server 340 may monitor the unique email address and determine whether an email received from a courier service tracking system indicates that a delivery is ready. For example, the server 340 may detect information in the “from” field, the subject line, and/or the content field of the email, to determine if the email indicates that a package is ready for delivery. Upon determining that an email received from one of the courier service tracking systems indicates that a package is ready for delivery, the server 340 may transmit a webhook to the API of the container 300 via Internet 310 and router 305, which causes the mechanical spring 230 to unlock so that the door 105 can be opened.

Regardless of which embodiment is used to open the door 105 of the container, at step 535, the controller may activate the audible buzzer 225 and/or the LED light 220 to emit a sound and/or light, respectively, to notify the driver of the container's presence. At step 540, the driver deposits the package inside the container, and at step 545 the driver manually closes the door 105. The controller receives a signal from mechanical spring lock 230 and/or a circuit coupled therebetween, indicating that the door 105 is closed, and the mechanical spring lock 230 is locked. At step 550, in response to receiving the signal that the door 105 is closed, the controller may determine whether it has received a signal from the motion sensor 215 indicating that motion has been detected in the container. If the controller has not received a signal indicating motion has been detected in the container, at step 555 the controller may activate the light emitting devices 205 and/or the ozone generator 210 to kill pathogens that may be inside the container, the outside of the package that was delivered, and/or inside the package. If the controller has received a signal indicating that motion has been detected inside the container, the controller will not allow the light emitting devices 205 or the ozone generator 210 to activate, and may activate the audible buzzer 225 and/or LED light 230 to indicate that movement has been detected in the container. At step 560, the controller may deactivate the light emitting devices 205 and/or the ozone generator 210 after a predetermined period sufficient to kill pathogens inside the container, and cause the screen 110 to display a notification that the sanitizing process is complete.

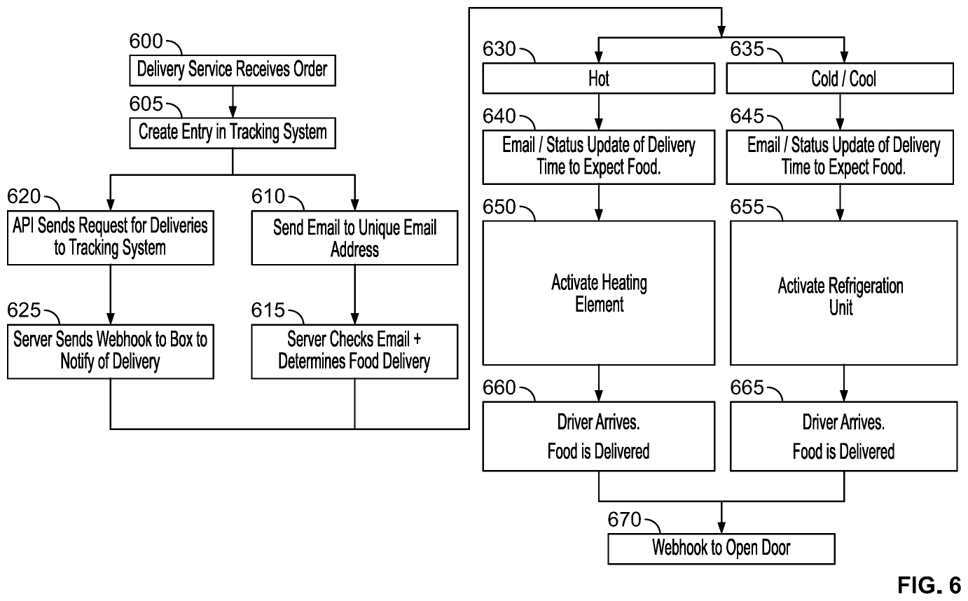

FIG. 6 depicts a flowchart showing a process for delivering temperature-sensitive packages, such as food items, to the container according to an exemplary embodiment. Referring to FIGS. 3 and 6, a food delivery tracking system 345, which may be associated with a restaurant or a third party delivery service, may receive an order for delivery at step 600, which may include the customer's name, delivery address, etc. At step 605, the food delivery tracking system 345 generates an entry for the order received at step 600. The entry may include a unique tracking number or identifier, as well as the customer's name, address, and other information related to the delivery order. The entry may further include the customer's email address and/or mobile phone number, as well as a unique email address corresponding to the container, which was generated when the customer registered the container. Alternatively, if the food delivery tracking system 345 already has an informed delivery profile for the customer, the food delivery tracking system 345 may use information received with the order in step 600, e.g., customer's name and/or address, to identify the informed delivery profile associated with the customer that placed the order. If an informed delivery profile is found within the food delivery tracking system 345, customer information contained within the informed delivery profile, such as the customer's email address, the unique email address and/or MAC ID associated with the customer's container, may be associated with the order. Alternatively, if the customer does not already have an informed delivery profile registered with the food delivery tracking system 345, the customer may create one when the customer submits the order.

At step 610, the food delivery tracking system 345 may transmit an email message to the unique email address associated with the customer's container, or otherwise transmit a notification to the container using the container's MAC ID, notifying the container that a food order has been received by the food delivery tracking system 345. At step 615, the server 340, which monitors the unique email address associated with the container, may receive the email sent by the food delivery tracking system 345 in step 610, and determine whether the email indicates delivery of a temperature-sensitive package. The server 340 may determine whether the package is temperature-sensitive, and whether the package should be received in a hot or cold environment, based on the name of the food delivery tracking system that sent the email, the subject line of the email, the content of the email, or other indicator contained in the email.

According to an alternative exemplary embodiment, rather than sending an email to the unique email address in step 610, after the entry for the order is created at step 605, the API of the container 300 may send a request to the food delivery tracking system 345 in step 620 to determine if any deliveries have been entered into the food delivery tracking system 345 for the address corresponding to the location of the container 300. In step 625, the food delivery tracking system 345 determines whether a delivery entry has been created corresponding to the location of the container 300, and if a delivery entry has been created, the food delivery tracking system 345 transmits a notification to the server 340, which transmits a webhook to the API of the container 300 indicating that a delivery has been found. For example, if the food delivery tracking system 345 includes an informed delivery profile corresponding to the container 300, the food delivery tracking system 345 may verify the MAC ID of the container 300, the delivery address, the customer name, or other identifying information to determine whether a delivery entry has been created for the container 300 that transmitted the request.

Upon determining, in either step 615 or 625, that a temperature-sensitive delivery entry has been created, the process proceeds to step 630 if the delivery entry needs to be kept warm, or to step 635 if the delivery entry needs to be kept cold. In step 640 or 645, depending on whether the delivery entry must be kept warm or cold, the food delivery tracking system 345 may transmit an email to the unique email address or transmit a webhook to the API of the container 300 indicating an expected delivery time. In the embodiment in which an email is transmitted to the unique email address, the server 340 may transmit a signal to the wireless chip of the PCB contained within the container to notify the container of the impending delivery of a temperature-sensitive package. In step 650, the container 300 may activate a heating element to warm the interior of the container 300 based on the estimated delivery time received in step 640. For example, the controller of the container 300 may be coupled to a heating to a heating element, and may activate the heating element a predetermined amount of time before or after the expected arrival time of the delivery. Alternatively, if the delivery must be kept cold, in step 655 the controller may activate a cooling element a predetermined amount of time before or after the arrival of the delivery to reduce the internal temperature of the container 300. The cooling element may include a peltier circuit, but other types of cooling elements may be used.

At step 660 or 665, the delivery driver arrives to deliver the temperature-sensitive package, and follows the manual delivery mode 510 or automatic delivery mode 520, as shown in FIG. 5. The controller may deactivate the heating or cooling element at the estimated delivery time, after a predetermined maximum time, or after the delivery driver indicates that the delivery is ready at step 511 or 521. The delivery process may thereafter continue as shown in FIG. 5.

According to an alternative exemplary embodiment, the heating or cooling element may be activated upon arrival of the delivery driver. For example, when the driver indicates that a package is ready for delivery in either step 511 or 521, the controller may activate the heating or cooling element in parallel with unlocking the door 105 of the container 300.

Although the inventive concepts of the present disclosure have been described and illustrated with respect to exemplary embodiments thereof, it is not limited to the exemplary embodiments disclosed herein and modifications may be made therein without departing from the scope of the inventive concepts.

What is claimed is:

1. A container for receiving packages, the container comprising:
a body configured to receive a package;
a door coupled to the body, said door having a locking mechanism configured to prevent the door from opening when engaged;
a controller configured to receive a signal corresponding to the package, and to unlock the locking mechanism in response to said signal; and
a sanitization device configured to sanitize at least one of the package and contents of the package by emitting at least one of a gas and a light configured to kill one or more of bacteria, viruses, and other pathogens.

2. The container of claim 1 further comprising a motion sensor configured to detect motion within the body of the container;
wherein the controller is configured to deactivate the sanitization device in response to the motion sensor detecting motion within the body of the container.

3. The container of claim 1, wherein the sanitization device comprises an ultraviolet light configured to emit light having a wavelength configured to kill one or more of bacteria, viruses, and other pathogens.

4. The container of claim 3, wherein the sanitization device further comprises an ozone generator configured to generate ozone gas configured to kill one or more of bacteria, viruses, and other pathogens.

5. The container of claim 1 further comprising an alarm;
wherein upon receipt of the signal corresponding to the package, the controller causes the alarm to emit a sound or vibration to alert a deliver person to the presence of the container.

6. The container of claim 1, further comprising a memory chip configured to store an identification number corresponding to package to be delivered to the container; and
a touchscreen configured to receive an input number;
wherein the signal to unlock the locking mechanism is generated based on the input number matching the identification number stored in the memory chip.

7. The container of claim 1 further comprising a memory chip configured to store identifying information corresponding to a package to be delivered to the container; and
a code reader configured to decode information encoded in a code on a package;
wherein the signal to unlock the locking mechanism is generated based on the information decoded by the code reader matching identifying information stored in the memory chip.

8. The container of claim 1, wherein the controller receives the signal to unlock the locking mechanism by receiving a webhook from a server;
wherein the server generates the webhook in response to a remote scanning device decoding information encoded in a code on a package, and said decoded information matching identifying information stored in a delivery tracking system for a package to be delivered to the container.

9. The container of claim 1, wherein the controller receives the signal to unlock the locking mechanism by receiving a webhook from a server;
wherein the server generates the webhook in response to receiving an email sent to a unique email address corresponding to the container indicating that a package is ready for delivery to the container;
wherein the email is generated by a delivery tracking system in response to receiving a signal from a remote scanning device containing information decoded from a code located on the package that matches identifying information stored in the delivery tracking system for a package to be delivered to the container.

10. The container of claim 1 further comprising at least one of a heating element configured to increase an interior temperature of the container and a cooling element configured to decrease the interior temperature of the container;
wherein the signal to unlock the locking mechanism includes a temperature signal to activate the heating element or cooling element; and
wherein the controller is configured to activate at least one of the heating element and cooling element based on the temperature signal.

11. A method of securely delivering a package to a container, the method comprising:
storing information corresponding to the package in a memory of the container;
receiving a signal indicating that the package is ready for delivery to the container;
unlocking a locking mechanism of the container in response to receiving the signal; and
activating a sanitization device to sanitize the package after the package has been placed inside the container.

12. The method of claim 11 further comprising determining whether motion occurs within the container;
wherein the activating of the sanitization device depends on whether motion is detected within the container.

13. The method of claim 11, wherein activating the sanitization device comprises emitting at least one of an ultraviolet light and an ozone gas configured to kill one or more of bacteria, viruses, and other pathogens.

14. The method of claim 11 further comprising activating an alarm to emit a sound or vibration in response to receiving the signal indicating that the package is ready for delivery to the container.

15. The method of claim 11, wherein said receiving a signal indicating the package is ready for delivery comprises receiving identifying information related to the package via an input device of the container; and
wherein said unlocking the locking mechanism comprises comparing the received identifying information to the information corresponding to the package stored in the memory of the container, and unlocking the locking mechanism based on whether the received identifying information corresponds to the information stored in the memory of the container.

16. The method of claim 16, wherein said input device comprises a code reader, and said identifying information related to the package comprises encoded information on the package that is decoded by the code reader.

17. The method of claim 11, wherein said receiving a signal indicating the package is ready for delivery comprises receiving a webhook from a server;
wherein said server generates the webhook in response to a remote scanning device decoding information encoded in a code on the package, and said decoded information matching identifying information stored in a delivery tracking system for a package to be delivered.

18. The method of claim 11, wherein said receiving a signal indicating the package is ready for delivery comprises receiving a webhook from a server;
wherein said server generates the webhook in response to receiving an email sent to a unique email address corresponding to the container indicating that a package is ready for delivery to the container;
wherein the email is generated by a delivery tracking system in response to receiving a signal from a remote scanning device containing information decoded from a code located on the package that matches identifying information stored in the delivery tracking system for a package to be delivered to the container.

19. The method of claim 11 further comprising activating at least one of a heating element and a cooling element based on receiving said signal indicating that the package is ready for delivery to the container.
